(19) United States
(12) Patent Application Publication
(10) Pub. No.: US 2004/0123347 A1
(43) Pub. Date: Jun. 24, 2004

(54) Water-deficit-inducible plant promoters

(76) Inventors: Brendan S. Hinchey, Mystic, CT (US); Robert J. Bensen, Niantic, CT (US); Donald E. Nelson, Stonington, CT (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/739,565

(22) Filed: Dec. 18, 2003

Related U.S. Application Data
(60) Provisional application No. 60/435,987, filed on Dec. 20, 2002.

(51) Int. Cl. A01H 1/00; C12N 15/82

(52) U.S. Cl. 800/279; 800/289

(57) ABSTRACT

The current invention provides the water-deficit-inducible promoters derived from the 5′ regulatory region of a HVA22, HSP17.5, RAB17 or CA4H genes. Such water-deficit-inducible promoters are operably linked to heterologous DNA of a gene of interest in a DNA construct which can be used for producing transgenic plants which can express a gene of interest under water-deficit induced conditions. The invention provides transgenic plants having such DNA constructs incorporated into their genome.

### CROSS REFERENCE TO RELATED APPLICATIONS

This application claims priority to U.S. provisional patent application serial No. 60/435,987 filed Dec. 20, 2002, which is incorporated herein by reference in its entirety.

### INCORPORATION OF SEQUENCE LISTINGS

A sequence listing is contained in the file named “SEQ ID for 38-15(53193)B.ST25.txt” which is 7.56 kilobytes (measured in MS-Windows) and was created on Dec. 11, 2003. The sequence listing is located in computer readable form on a 3.5 inch diskette filed herewith and incorporated herein by reference.

### BACKGROUND OF THE INVENTION

This invention discloses water-deficit-inducible plant promoters, DNA constructs with such promoters, transgenic plants with such promoters, and DNA constructs and methods of making and using such promoters, DNA constructs and transgenic plants.

Water-deficit can have adverse effects on plants such as yield reductions, increased susceptibility to disease and pests, reduced plant growth and reproductive failure. An object of this invention is to provide plants which can express genes to ameliorate the adverse effects of water-deficit. Useful genes for expression especially during water-deficit are genes which promote aspects of plant growth or fertility, genes which impart disease resistance, genes which impart pest resistance, stress-responsive transcription factors and the like.

Considering the complexity of water use in land plants, especially during conditions that produce water-deficit, relatively few promoters specifically associated with this aspect of physiology have been identified. It would be of benefit to the art to increase the number and variety of promoters involved in regulating water use in plants, more particularly, in corn plants, and even more particularly in corn plants experiencing water-deficit. It would be especially advantageous to identify promoters which can be used in directing the expression of genes which are beneficial to the plant when induced during water-deficit, while having low to no expression under adequately watered conditions.

### SUMMARY OF THE INVENTION

This invention provides water-deficit-inducible promoters for use in plants which are derived from the 5′ regulatory region of maize genes identified as a heat shock protein 17.5 gene (HSP17.5), an HVA22 gene (HVA22), a cinnamic acid 4-hydroxylase (CA4H) gene (CA4H), and a rab17 gene (RAB17).

In one embodiment of the invention the promoters are derived from the 5′ regulatory region of maize HSP17.5 and have a nucleotide sequence identical to part or all of SEQ ID NO:1. In another embodiment of the invention the promoters are derived from the 5′ regulatory region of maize HVA22 and have a nucleotide sequence identical to part or all of SEQ ID NO:2. In yet another embodiment of the invention the promoters are derived from the 5′ regulatory region of maize CA4H and have a nucleotide sequence identical to part or all of SEQ ID NO:3. In another embodiment of the invention the promoters are derived from the 5′ regulatory region of maize RAB17 and have a nucleotide sequence identical to part or all of SEQ ID NO:5 comprising at least the nucleotides at positions 156 and 157.

One aspect of this invention provides water-deficit-inducible promoters which exhibit promoter activity in plant tissue having a water potential of less than −0.7 megaPascals (MPa), e.g. at less than −0.8 MPa or lower, such as less than −0.9 MPa or −0.10 MPa.

This invention provides DNA constructs with water-deficit-inducible promoters for expressing heterologous DNA in plants during water-deficit, methods for providing plants which express DNA of interest during water-deficit, and transgenic plants having such DNA constructs. One aspect of the invention provides a DNA construct comprising a promoter operably linked to a heterologous DNA, where the promoter is derived from part or all of an HVA22, HSP17.5, RAB17, or CA4H gene and exhibits promoter activity under water-deficit-inducible conditions. Another aspect of the invention provides such DNA constructs where the promoter comprises at least 100 contiguous nucleotides which are identical to a nucleic acid sequence of SEQ ID NOS:1-3, or a larger identical segment, e.g., about 125 nucleotides, about 500 nucleotides ore more. Another aspect of the invention provides such DNA constructs where the promoter comprises at least 100 contiguous nucleotides which are identical to a nucleic acid sequence of SEQ ID NO:5, or a larger identical segment, e.g., about 125 nucleotides, about 500 nucleotides or more, where the fragment comprises base pairs 156 and 157 of SEQ ID NO:5.

Still other aspects provide such DNA constructs where the promoter comprises a segment of nucleic acid sequence which is at least 85% identical with a functional part of SEQ ID NOS:1-3, e.g. at least a 100 contiguous nucleotide segment of SEQ ID NOS:1-3 or a larger fragment of the natural promoter, e.g. at least about 250, or 500, or even 1000 contiguous nucleotide segment. Other aspects of the invention provide such DNA constructs where the promoter comprises the CAAT and TATA box elements naturally associated with the natural promoter. Yet another aspect of the invention provides such DNA constructs where the water-deficit-inducible promoter is operably linked to a heterologous DNA which encodes a molecule imparting enhanced environmental stress tolerance, e.g. water-deficit tolerance.

Another aspect of this invention provides a method for providing a plant which expresses a gene of interest under water-deficit conditions comprising introducing into the genome of the plant a DNA construct comprising a water-deficit-inducible promoter operably linked to heterologous DNA desired to be expressed during water-deficit. Preferably such a plant has a water-deficit-inducible promoter which exhibits promoter activity in plant tissue having a water potential of less than about −0.7 MPa, e.g. at less than about −0.8 MPa or lower, such as less than about −0.9 MPa or about −0.10 MPa.

A further aspect of this invention provides transgenic plants with a water-deficit-inducible promoter operably linked to heterologous DNA, e.g. a gene of interest. More particularly such a transgenic plant has in its genome a DNA construct according to this invention, e.g. a DNA construct comprising a promoter operably linked to heterologous DNA where the promoter is derived from the 5′ regulatory region of an HVA22, HSP17.5, RABI 7, or CA4H gene. The transgenic plant may be a monocot or a dicot. A preferred monocot transgenic plant may be selected from the group consisting of wheat, oat, barley, maize, rye, rice, turfgrass, sorghum, millet and sugarcane, more preferably maize, wheat and rice. A preferred dicot transgenic plant may be selected from the group consisting of canola, cotton, safflower, soybean, sugarbeet, sunflower, more preferably soybean, canola and cotton.

The present invention also provides for a transgenic plant comprising a water-deficit-inducible promoter in combination with an enhancer, for example, an intron. In one embodiment, the enhancer intron is a rice actin 1 intron 1 (U.S. Pat. No. 5,641,876, incorporated herein by reference in its entirety) or a rice actin 2 intron 1 (U.S. Pat. No. 6,429,357, incorporated herein by reference in its entirety). The promoter element may further comprise a 3′ untranslated region (3′ UTR), such as a nos or Tr7 3′ UTR.

In one embodiment of the invention, the transgenic plant comprises a DNA construct with a selected heterologous DNA operably linked to a native or exogenous water-deficit-inducible promoter or a derivative of a native or exogenous water-deficit-inducible promoter. Preferred heterologous DNA includes genes that are effective in or needed by a plant during water-deficit conditions for plant growth or survival. Potentially any heterologous DNA can be operably linked to the water-deficit-inducible promoter, including a selected sequence which encodes a molecule imparting insect resistance, bacterial disease resistance, fungal disease resistance, viral disease resistance, nematode disease resistance, herbicide resistance, enhanced grain composition or quality, nutrient transporter functions, enhanced nutrient utilization, enhanced environmental stress, reduced mycotoxin contamination, female sterility, a selectable marker phenotype, a screenable marker phenotype, a negative selectable marker phenotype, altered plant agronomic characteristics, a stress-responsive transcription factor or a combination thereof. In preferred aspects of this invention the water-deficit-inducible promoter is operably linked to heterologous DNA which encodes a molecule imparting enhanced environmental stress tolerance, e.g. water-deficit tolerance.

The selected heterologous DNA may further comprise DNA from a cloning vector, such as plasmid DNA, or alternatively, may have been introduced as an expression cassette isolated from such vector DNA. The selected DNA may also comprise a sequence encoding a signal peptide. Examples of signal peptides that could be used include a peroxisomal targeting peptide or a chloroplast transit peptide. Examples of a chloroplast transit peptide include the group consisting of chlorophyll a/b binding protein transit peptide, small subunit of ribulose bisphosphate carboxylase transit peptide, EPSPS transit peptide and dihydrodipocolinic acid synthase transit peptide.

A transgenic plant prepared in accordance with the invention may be of any generation, including a fertile R₀ transgenic plant as well as progeny plants of any generation and hybrid progeny plants thereof which contain the heterologous DNA. Also included within the invention are seeds of any such plants.

In still yet another aspect, the invention provides a method of plant breeding comprising the steps of: (i) obtaining a transgenic plant comprising a water-deficit-inducible promoter of this invention and (ii) crossing the transgenic plant with itself or a second plant. The transgenic plant may be of potentially any species, including monocotyledonous or dicotyledonous plants.

### BRIEF DESCRIPTION OF THE DRAWINGS

FIG. 1 is 1316 bp of DNA sequence comprising a 5′ regulatory region of a maize HSP17.5 gene (SEQ ID NO:1).

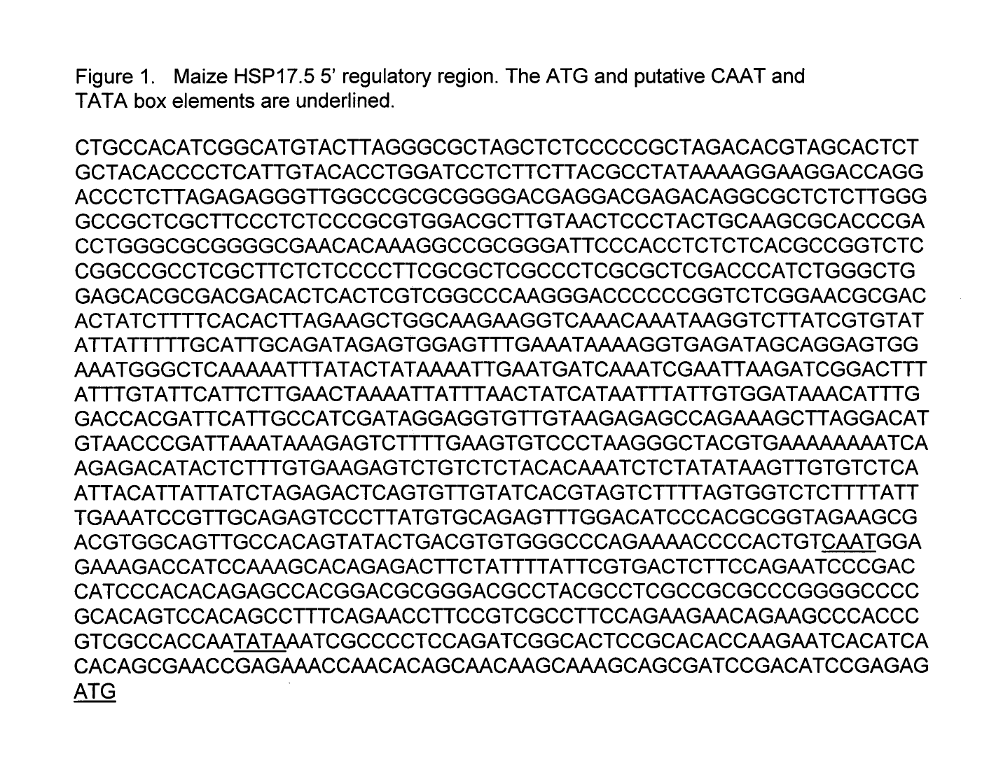

FIG. 2 is 764 bp of DNA sequence comprising a 5′ regulatory region of a maize HVA22 gene (SEQ ID NO:2).

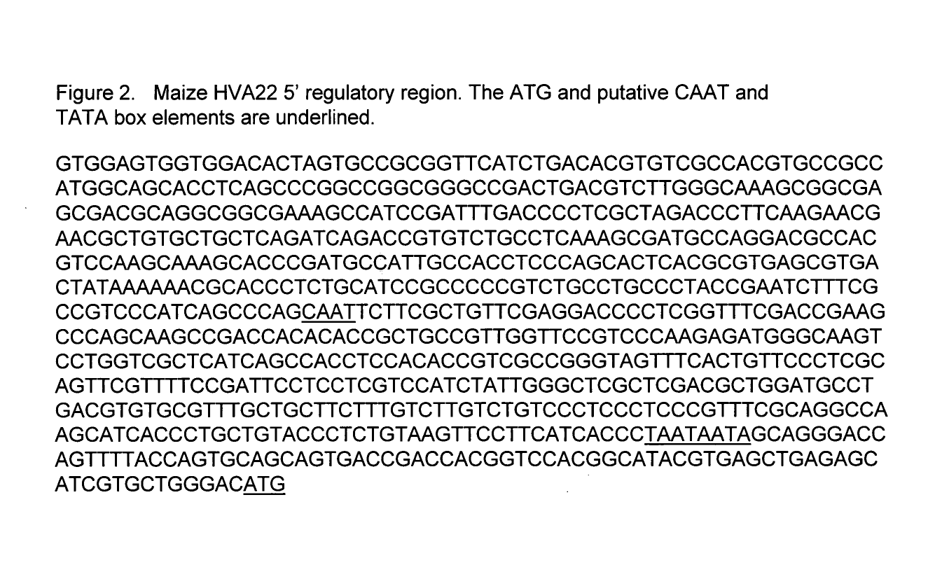

FIG. 3 is 1310 bp of DNA sequence comprising a 5′ regulatory region of a maize CA4H gene (SEQ ID NO:3).

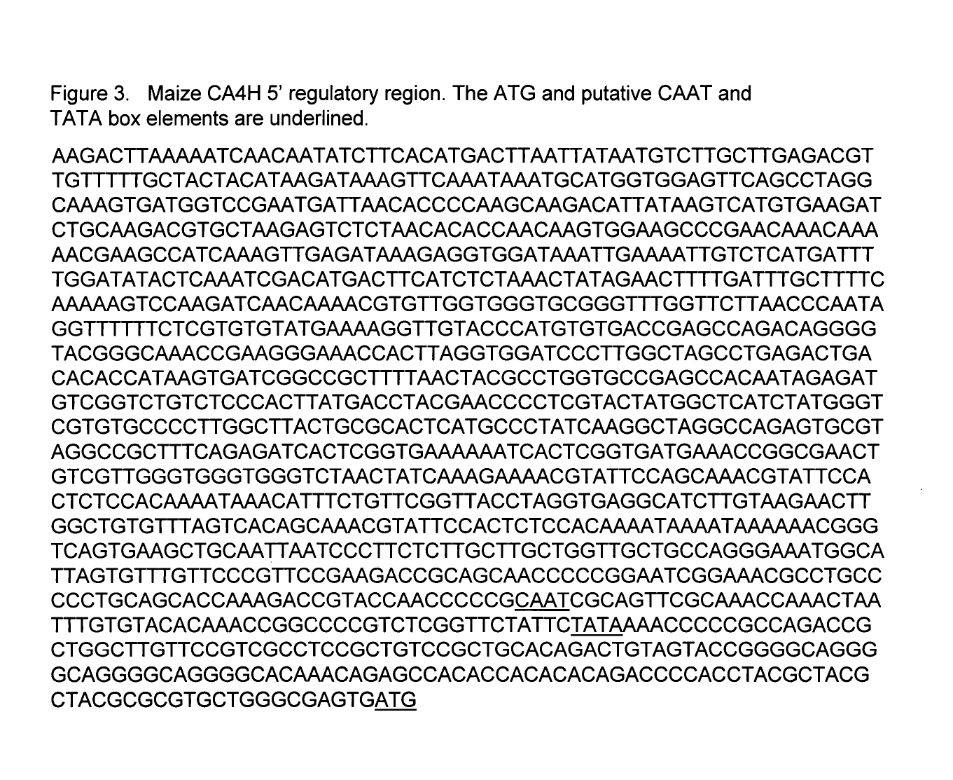

FIG. 4 is 574 bp of DNA sequence comprising a 5′ regulatory region of a maize RAB17 gene (SEQ ID NO:4) disclosed by Vilardell et al., Plant Molecular Biology, 17(5):985-993, 1990.

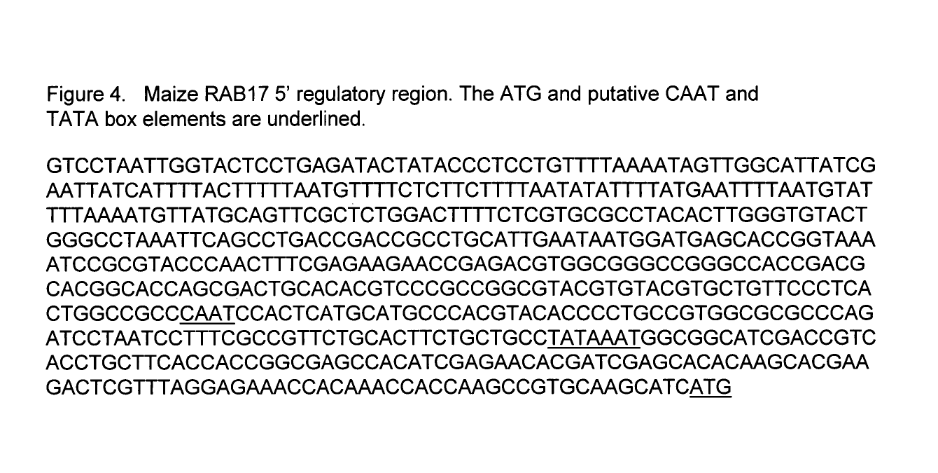

FIG. 5 is 571 bp of DNA sequence comprising a 5′ regulatory region of a maize RAB17 gene (SEQ ID NO:5).

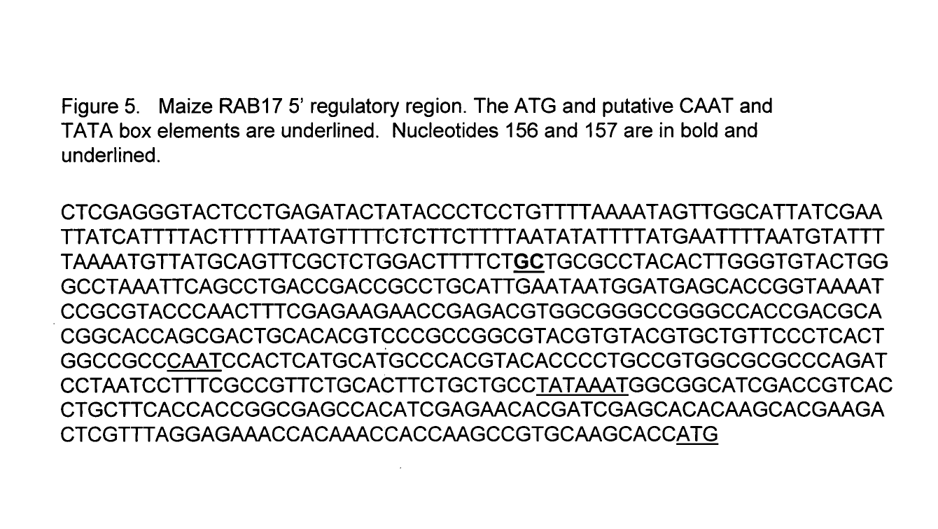

FIG. 6 illustrates vector pMON78018 which contains a water-deficit-inducible promoter of the invention.

FIG. 7 illustrates vector pMON78019 which contains a water-deficit-inducible promoter of the invention.

### DETAILED DESCRIPTION OF THE INVENTION

### 1. DEFINITIONS

As used herein “coding sequence” means a DNA sequence which is a template for the production of an RNA molecule. The RNA may be an mRNA, which encodes a protein product, or a tRNA, rRNA, snRNA, antisense RNA, or other RNA molecule, such as a hairpin-forming RNA which is useful for gene suppression.

As used herein “exogenous DNA” refers to DNA which is not normally found next to the adjacent native DNA, i.e., a sequence not normally found in the host genome in an identical context. The DNA itself may be native to the host genome or may comprise the native sequence altered by the addition or deletion of one or more different regulatory elements or other sequences. The exogenous DNA may encode a protein or non-protein product. Likewise, “exogenous sequence” is a sequence of DNA not normally found in the host genome in an identical context. A transformation construct comprising a gene of interest, which originates or is produced outside of an organism, is an example of an exogenous DNA.

As used herein “expression” refers to the combination of intracellular processes, including transcription and translation, undergone by a DNA molecule, such as a structural gene to produce a polypeptide, or a non-structural gene to produce an RNA molecule.

As used herein “gene” means a DNA sequence from which an RNA molecule is transcribed. The RNA may be an mRNA which encodes a protein product, an RNA which functions as an anti-sense molecule, or a structural RNA molecule such as a tRNA, rRNA, snRNA, or other RNA.

As used herein “heterologous” DNA is any DNA sequence which is not naturally found next to the adjacent DNA. Heterologous DNA is often found in a DNA construct used for transformation. A water-deficit-inducible promoter of the instant invention, e.g. rab17, operably linked to a reporter gene is an example of a heterologous DNA as the rab17 promoter is naturally and normally associated with a rab17 gene.

As used herein “progeny” means any subsequent generation, including the seeds and plants therefrom, which is derived from a particular parental plant or set of parental plants; the resultant progeny line may be inbred or hybrid. Progeny of a transgenic plant of this invention can be, for example, self-crossed, crossed to a transgenic plant, crossed to a non-transgenic plant, and/or back crossed.

As used herein, “promoter” means a region of DNA sequence that is essential for the initiation of transcription of DNA, resulting in the generation of an RNA molecule that is complimentary to the transcribed DNA; this region may also be referred to as a “5′ regulatory region.” Promoters are located upstream of the coding sequence to be transcribed and have regions that act as binding sites for RNA polymerase and have regions that work with other factors to promote RNA transcription. More specifically, basal promoters in plants comprise canonical regions associated with the initiation of transcription, such as CAAT and TATA boxes. The TATA box element is usually located approximately 20 to 35 nucleotides upstream of the site of initiation of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is regions comprising sequences from the CAAT box to the translational start site, with other upstream promoter elements to enhance or otherwise alter promoter activity or specificity.

As used herein “promoter activity” characterizes a DNA sequence which initiates transcription of RNA from adjacent downstream DNA.

As used herein an “Ro transgenic plant” is a plant which has been directly transformed with a selected DNA or has been regenerated from a cell or cell cluster which has been transformed with a selected DNA.

As used herein “regeneration” means the process of growing a plant from a plant cell (e.g., plant protoplast, callus or explant).

As used herein “transformation construct” means a DNA molecule which is designed for introduction into a host genome by genetic transformation. Preferred transformation constructs will comprise all of the genetic elements necessary to direct the expression of one or more exogenous sequences. Transformation constructs prepared in accordance with the instant invention will include a promoter derived from a maize HSP17.5, HVA22, CA4H or RAB17 promoter. In particular embodiments of the instant invention, it may be desirable to introduce a transformation construct into a host cell in the form of an exogenous DNA construct.

As used herein “transgene” means a segment of DNA which has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

As used herein “transgenic plant” means a plant or progeny plant of any subsequent generation derived therefrom, wherein the DNA of the plant or progeny thereof contains an introduced exogenous DNA segment not originally present in a non-transgenic plant of the same strain. The transgenic plant may additionally contain sequences which are native to the plant being transformed, but wherein the “exogenous” gene has been altered in order to alter the level or pattern of expression of the gene.

As used herein a “stably” transformed plant is a plant in which the exogenous DNA is heritable. The exogenous DNA may be heritable as a fragment of DNA maintained in the plant cell and not inserted into the host genome. Preferably, the stably transformed plant comprises the exogenous DNA inserted into the chromosomal DNA in the nucleus, mitochondria or chloroplast, most preferably in the nuclear chromosomal DNA.

As used herein “water-deficit” is a plant condition characterized by water potential in a plant tissue of less than about −0.7 MPa, e.g. about −0.8 Mpa. Water potential in maize is conveniently measured by clamping a leaf segment in a pressurizable container so that a cut cross section of leaf is open to atmospheric pressure. Gauge pressure (above atmospheric pressure) on the contained leaf section is increased until water begins to exude from the atmospheric-pressure-exposed cross section; the gauge pressure at incipient water exudation is reported as negative water potential in the plant tissue, e.g. 0.7 MPa gauge pressure is reported as −0.7 MPa water potential.

### 2. WATER-DEFICIT-INDUCIBLE PROMOTERS

This invention provides novel methods and DNA constructs expressing transgenes in plants, especially under conditions that produce water-deficit in plants. Plants from a number of maize lines were field-grown under non-irrigated (water-deficit-producing) or irrigated conditions. Leaf samples were taken from plants before the tassel stage for each field condition to allow measurement of water potential and isolation of RNA. RNA from water-deficit and non-water-deficit samples was analyzed for differences using transcriptional profiling array methods. A number of RNAs were found to show differences in accumulation, to either higher or lower levels in the plants, depending upon the water treatment.

Array samples were selected that demonstrated about a 3 to 8 fold increase in RNA accumulation in water-deficit plants. The water-deficit-inducible maize genes were identified as a heat shock protein 17.5 (HSP17.5) gene, an HVA22 gene, a gene encoding cinnamic acid 4-hydroxylase (CA4H) and a RAB17 gene. HSP17.5 is one of a number of low molecular weight heat shock proteins identified in plants. Heat shock genes, first identified by response to elevated temperatures in Drosophila, have been identified in a wide variety of organisms. HVA22 is one of many stress-induced genes known in plants, and homologues of this gene have been described in barley, Arabidopsis and other plants. HVA22 has been described as being responsive to abscisic acid (ABA); promoter analysis of barley clones has identified a number of ABA response elements, as well as other associated sequences in the promoter, which affect and allow for hormonal response. CA4H is a member of the cytochrome P450 monooxygenase superfamily. It is thought to play a role in phenylpropanoid metabolism and lignin biosynthesis in plants. Promoters have been isolated for Aiabidopsis CA4H, and while a number of putative cis-acting elements have been identified, matches to such elements are not evident in the promoter sequence of maize CA4H which is useful in this invention. RAB17 is a gene isolated from maize that is ABA responsive as well as water-deficit responsive (Vilardell et al., Plant Molecular Biology, 17(5):985-993, 1990).

The regulatory promoter regions isolated from the maize HVA22, HSP17.5, RAB17 or CA4H genes are useful in defining promoters for this invention. Under well-watered conditions, these promoters drive expression of the native genes to very low or non-detectable levels. Water-deficit-inducible promoters derived from the 5′ regulatory region of maize HVA22, HSP17.5, RAB17 or CA4H genes can be used in directing the expression of potentially any gene which one desires to have expressed when water is limiting during plant growth.

These promoters represent a significant advance in that they are capable of directing water-deficit-regulated expression of transgenes. The specific, inducible nature of the promoters of the invention is important in that it allows expression of a transgene operatively linked to the promoter under conditions of water-deficit with little to no expression under well-watered conditions. By avoiding continuous high-level expression of transgenes, any undesired effects, e.g. diminished traits such as yield drag sometimes associated with transgene expression by a constitutive promoter, caused by continual over-expression of transgenes, or ectopic expression in various tissues or at various times, can be minimized or eliminated.

The HVA22, HSP17.5, RAB17 or CA4H promoter sequences useful in the various aspects of this invention can be derived from any plant. Embodiments of promoter sequences which were isolated from maize to illustrate this invention have nucleic acid sequences given in SEQ ID NO:1 (HSP17.5), SEQ ID NO:2 (HVA22), SEQ ID NO:3 (CA4H), SEQ ID NO:4 (RAB17) and SEQ ID NO:5 (RAB17). A putative TATA box element is identified as beginning at about nucleotide 1205 and a putative CAAT box element is identified as beginning at about nucleotide 1011 in SEQ ID NO:1. A putative TATA box element is identified as beginning at about nucleotide 674 and a putative CAAT box element is identified as beginning at about nucleotide 359 in SEQ ID NO:2. A putative TATA box element is identified as beginning at about nucleotide 1151 and a putative CAAT box element is identified as beginning at about nucleotide 1088 in SEQ ID NO:3. A putative TATA box element is identified as beginning at about nucleotide 444 and a putative CAAT box element is identified as beginning at about nucleotide 360 in SEQ ID NO:5.

In addition to the unmodified HVA22, HSP17.5, RAB17 or CA4H promoter containing DNA sequences of SEQ ID NOS:1-3 and 5, the current invention includes certain derivatives of these DNA sequences and compositions made therefrom. One important application of the HVA22, HSP17.5, RAB17 or CA4H promoters is in the construction of DNA constructs designed for introduction into plants by genetic transformation.

### 3. DERIVATIVE WATER-DEFICIT-INDUCIBLE PROMOTERS

This invention provides water-deficit-inducible promoters which have been derived from the 5′ regulatory regions of the HVA22, HSP17.5, RAB17 or CA4H genes. Derivatives of these promoters may include, but are not limited to, deletions of sequence, single or multiple point mutations, alterations at a particular restriction enzyme site, addition of functional elements, or other means of molecular modification which may enhance, or otherwise alter promoter expression. Derivative promoters prepared from a water-deficit-inducible promoter of the invention may exhibit, for example, alterations in patterns of expression, levels of expression, timing, responsiveness to stress or water-deficit, tissue specificity, combinations of these variations or even other alterations. For example, one of skill in the art may delimit the functional elements within the HVA22, HSP17.5, RAB17 or CA4H promoters and delete any non-essential elements. Functional elements may be modified or combined to increase the utility or expression of the sequences of the invention for any particular application. For example, a functional region within the HVA22, HSP17.5, RAB17 or CA4H promoters of the invention could be modified to decrease or increase inducible expression. The means for mutagenizing or creating deletions in a DNA segment encoding HVA22, HSPI 7.5, RAB17 or CA4H promoter sequence of the current invention are well-known to those of skill in the art and are disclosed, for example, in U.S. Pat. No. 6,583,338, incorporated herein by reference in its entirety.

It is anticipated that fragments of natural HVA22, HSP17.5, RAB17 or CA4H promoters that are especially useful for allowing promoter functionality include, but are not limited to:

(1) the 5′ UTR region from the transcriptional start site to the ATG, the about 300 nucleotide base pair 5′ UTR region from the CAAT box to the ATG, including the TATA box (about nucleotide 1012 to about 1313 of SEQ ID NO:1), from the CAAT box to the transcriptional start site, and all sequences and fragments thereof upstream of the CAAT box (from about nucleotide 1 to about 1011 of SEQ ID NO:1) of the maize HSP17.5 promoter;

(2) the 5′ UTR region from the transcriptional start site to the ATG, the about 400 nucleotide base pair 5′ UTR region from the CAAT box to the ATG, including the TATA box (about nucleotide 360 to about 761 of SEQ ID NO:2), from the CAAT box to the transcriptional start site, and all sequences and fragments thereof upstream of the CAAT box (from about nucleotide 1 to about 359 of SEQ ID NO:2) of the maize HVA22 promoter or,

(3) the 5′ UTR region from the transcriptional start site to the ATG, the about 220 nucleotide base pair 5′ UTR region from the CAAT box to the ATG, including the TATA box (about nucleotide 1089 to about 1307 of SEQ ID NO:3), from the CAAT box to the transcriptional start site, and all sequences and fragments thereof upstream of the CAAT box (from about nucleotide 1 to about 1088 of SEQ ID NO:3) of the maize CA4H promoter.

(4) the 5′ UTR region from the transcriptional start site to the ATG, the about 210 nucleotide base pair 5′ UTR region from the CAAT box to the ATG, including the TATA box (about nucleotide 360 to about 568 of SEQ ID NO:5), from the CAAT box to the transcriptional start site, and all sequences and fragments thereof upstream of the CAAT box (from about nucleotide 1 to about 359 of SEQ ID NO:5) of the maize RAB17 promoter.

The various promoter fragments described may be operably linked to a heterologous DNA in a DNA construct and used for plant transformation. Exogenous DNA comprising a marker gene or reporter gene is useful for testing the promoter activity of the various fragments. It is also anticipated that the sequences and fragments thereof upstream of the CAAT box (from about nucleotide 1 to about 1011 of SEQ ID NO:1; from about nucleotide 1 to about 359 of SEQ ID NO:2; from about nucleotide 1 to about 1088 of SEQ ID NO:3; from about nucleotide 1 to about 359 of SEQ ID NO:5) may be operably linked to heterologous CAAT and TATA boxes or other transcriptional start site sequences and exhibit promoter activity similar or identical to that of the full natural promoters.

Thus, promoters of this invention are not required to have 100% sequence identity to SEQ ID NO:1, SEQ ID NO:2 or SEQ ID NO:3. For instance, effective promoters can range from about 85% to 100% identity to SEQ ID NO:1 SEQ ID NO:2 or SEQ ID NO:3, or a fragment of SEQ ID NO:1, SEQ ID NO:2 or SEQ ID NO:3, e.g. a DNA fragment of at least 100 nucleotide base pairs, or larger, e.g. about 150, about 250, about 750 or even more nucleotide base pairs. In one aspect of the invention the promoters and derivative promoters are characterized as having at least 85% sequence identity, more preferably at least 90% sequence identity or higher, e.g. at least 95% or even 98% sequence identity with SEQ ID NO:1, SEQ ID NO:2 or SEQ ID NO:3, or such a fragment thereof.

### 4. PLANT TRANSFORMATION CONSTRUCTS

The construction of vectors which may be employed in conjunction with plant transformation techniques according to the invention will be known to those of ordinary skill in the art in light of this disclosure. Many approaches or methods have been developed and used for gene cloning. Examples of these are cloning by restriction enzyme digestion and ligation of compatible ends, T-A cloning directly from PCR product, TOPO-attached unidirectional cloning, and recombination-based cloning.

The techniques of the current invention are thus not limited to any particular DNA sequences in conjunction with a maize HVA22, HSP17.5, RAB17, or CA4H promoter of the invention. For example, an HVA22, HSP17.5, RAB17, or CA4H promoter alone could be transformed into a plant with the goal of enhancing or altering the expression of one or more genes in the host genome, particularly under water-deficit or reduced water conditions. Useful heterologous DNA sequences to operably link to the promoter sequences of the invention are exemplified by sequence encoding proteins, polypeptide products, RNA molecules such as antisense RNA molecules, marker genes, or combinations thereof. In certain embodiments, the present inventors contemplate the transformation of a recipient cell with more than one transformation construct, a co-transformation. Preferred components likely to be included with vectors used in the current invention are as follows: regulatory elements including 3′ unstranslated regions, 5′ untranslated regions, enhancers, introns, signal peptide coding sequences, transit peptide coding sequences, selectable marker genes, screenable marker genes, and the like.

A discussion of useful plant transformation constructs which can be prepared by those of ordinary skill in the art can be found among, for example, U.S. Pat. No. 5,641,876 which discloses a rice actin promoter, U.S. Pat. No. 6,429,357 which discloses a rice actin 2 promoter and intron, U.S. Pat. No. 6,084,089 which discloses cold inducible promoters, U.S. Pat. No. 6,140,078 which discloses salt inducible promoters, U.S. Pat. No. 6,252,138 which discloses pathogen inducible promoters, and U.S. patent application Publication Ser. No. 2002/0192813A1 which discloses 5′, 3′ and intron elements useful in the design of effective plant expression vectors, all of which are incorporated herein by reference.

In certain embodiments of the invention, transformation of a recipient cell may be carried out with more than one exogenous DNA. The DNA molecules may code for a protein product, or a non-protein product, such as a transfer RNA, anti-sense RNA or hairpin-forming RNA molecule. Two or more exogenous coding sequences can be supplied in a single transformation event using either distinct heterologous DNA vectors, or using a single vector incorporating two or more heterologous DNA sequences.

### 5. EXOGENOUS GENES FOR MODIFICATION OF PLANT PHENOTYPES

This invention provides plants which can express genes to counteract or ameliorate water-deficit. Useful genes for expression especially during water-deficit are genes which promote aspects of plant growth or fertility, genes which impart disease resistance, genes which impart pest resistance, and the like. The promoters of this invention which can express genes at a useful level during water-deficit with little if any expression during non-water-deficit conditions are useful for making such plants. In particular, the current invention provides promoters derived from the 5′ regulatory region of an HVA22, HSP17.5, RAB17, or CA4H gene for the expression of selected heterologous DNA in plants.

The choice of a selected DNA for expression in a plant host cell in accordance with the invention will depend on the purpose of the transformation. One of the major purposes of transformation of crop plants is to express any gene which one desires to have expressed when water is limiting during plant growth for imparting a commercially desirable, agronomically important or end-product traits to the plant. Such traits include, but are not limited to, herbicide resistance, herbicide tolerance, insect resistance, insect tolerance, disease resistance, disease tolerance (viral, bacterial, fungal, nematode), stress tolerance, stress resistance, as exemplified by resistance or tolerance to water-deficit, heat, chilling, freezing, excessive moisture, salt stress and oxidative stress, increased yield, food content and value, increased feed content and value, physical appearance, male sterility, female sterility, drydown, standability, prolificacy, starch quantity and quality, oil quantity and quality, protein quality and quantity, amino acid composition, and the like. It is also anticipated that expression of heterologous DNA encoding antisense RNAs or other RNA molecules are included as useful means for modifying plant phenotype. An especially useful class of selected DNA for use with the promoters of this invention are genes which encode a molecule which enhances environmental stress tolerance, e.g. genes which enhance water-deficit resistance or tolerance when expressed under water-deficit conditions.

Alternatively, an exogenous DNA sequence may be designed to down-regulate a specific nucleic acid sequence. This is typically accomplished by operably linking with a promoter, such as a water-deficit-inducible promoter of the invention, an exogenous DNA in an antisense orientation or a DNA designed such that a hairpin-forming RNA molecule is generated upon transcription. Gene suppression may be effective against a native plant gene associated with a trait, e.g. to provide plants with enhanced tolerance to water-deficit conditions. For example, a CA4H promoter of the invention may be operably linked to a heterologous DNA designed such that a hairpin-shaped RNA is formed for suppression of a native gene in maize embryos.

As used herein “gene suppression” means any of the well-known methods for suppressing an RNA transcript or production of protein translated from an RNA transcript, including post-transcriptional gene suppression and transcriptional suppression. Post-transcriptional gene suppression is mediated by double-stranded RNA having homology to a gene targeted for suppression. Gene suppression by RNA transcribed from an exogenous DNA construct comprising an inverted repeat of at least part of a transcription unit is a common feature of gene suppression methods known as anti-sense suppression, co-suppression and RNA interference. Transcriptional suppression can be mediated by a transcribed double-stranded RNA having homology to promoter DNA sequence to effect what is called promoter trans-suppression.

More particularly, post transcriptional gene suppression by inserting an exogenous DNA construct with anti-sense oriented DNA to regulate gene expression in plant cells is disclosed in U.S. Pat. No. 5,107,065 and U.S. Pat. No. 5,759,829, each of which is incorporated herein by reference in its entirety. Transgenic plants transformed using such anti-sense oriented DNA constructs for gene suppression can comprise DNA arranged as an inverted repeat, as disclosed by Redenbaugh et al. in “Safety Assessment of Genetically Engineered Flavr Savr™ Tomato, CRC Press, Inc. (1992). Inverted repeat insertions can comprises a part or all of a T-DNA construct.

Post transcriptional gene suppression by inserting an exogneous DNA construct with sense-oriented DNA to regulate gene expression in plants is disclosed in U.S. Pat. No. 5,283,184 and U.S. Pat. No. 5,231,020, each of which is incorporated herein by reference.

Different types of exogenous DNA arrangements resulting in gene suppression are known to those of skill in the art and include but are not limited to the following. International Publication WO 94/01550 discloses DNA constructs where the anti-sense RNA was stabilized with a self-complementary 3′ segment. Other double-stranded hairpin-forming elements in transcribed RNA are disclosed in International Publication No. 98/05770 where the anti-sense RNA is stabilized by hairpin forming repeats of poly(CG) nucleotides and patent application Publication Ser. No. 2002/0048814 A1 describes sense or anti-sense RNA stabilized by a poly(T)-poly(A) tail. U.S. patent application Publication Ser. No. 2003/0018993 A1 disclose sense or anti-sense RNA is stabilized by an inverted repeat of a subsequence of 3′ untranslated region of the NOS gene. U.S. patent application Publication Ser. No. 2003/0036197 A1 describe an RNA stabilized by two complementary RNA regions having homology to a target sequence.

Gene silencing can also be effected by transcribing RNA from both a sense and an anti-sense oriented DNA, e.g. as disclosed. in U.S. Pat. No. 5,107,065 and other examples as follows. U.S. Pat. No. 6,326,193 discloses gene targeted DNA which is operably linked to opposing promoters. Sijen et al., (The Plant Cell, Vol. 8, 2277-2294 (1996)) disclose the use of constructs carrying inverted repeats of a cowpea mosaic virus gene in transgenic plants to mediate virus resistance. Such constructs for post transcriptional gene suppression in plants by double-stranded RNA are also disclosed in International Publication No. WO 99/53050, International Publication No. WO 99/49029, U.S. patent application Publication Ser. No. 2003/0175965 A1, U.S. patent application Ser. No.10/465,800 and U.S. Pat. No. 6,506,559. See also U.S. application Ser. No. 10/393,347 which discloses constructs and methods for simultaneously expressing one or more recombinant genes while simultaneously suppressing one or more native genes in a transgenic plant. See also U.S. Pat. No. 6,448,473 which discloses multigene suppression vectors for use in plants. All of the above-described patents, applications and international publications disclosing materials and methods for post transcriptional gene suppression in plants are incorporated herein by reference.

Transcriptional suppression such as promoter trans suppression can be effected by a expressing a DNA construct comprising a promoter operably linked to inverted repeats of promoter DNA for a target gene. Constructs useful for such gene suppression mediated by promoter trans suppression are disclosed by Mette et al., (The EMBO Journal, Vol. 18, No. 1, pp. 241-148, 1999) and by Mette et al., (The EMBO Journal, Vol. 19, No. 19, pp. 5194-5201-148, 2000), both of which are incorporated herein by reference.

Preferable target nucleic acid sequences may encode proteins which are engaged in a plant during times of water excess. For example, it may be desirable to suppression genes involved in the opening of stomates. Alternatively, it may adventitious to suppress genes involved in the breakdown of particular osmotic protectants.

### 6. ASSAYS OF TRANSGENE EXPRESSION

To confirm the presence of an exogenous DNA in regenerated plants, a variety of assays may be performed. Such assays include, for example, molecular biological assays such as Southern and Northern blotting and PCR; biochemical assays such as detecting the presence of a protein product, e.g., by immunological means (ELISAs and Western blots) or by enzymatic function; plant part assays such as leaf or root assays; and in some cases phenotype analysis of a whole regenerated plant. Additional assays useful for determining the efficiency of transgene expression and promoter function also include without limitation fluorescent in situ hybridization (FISH), direct DNA sequencing, pulsed field gel electrophoresis (PFGE) analysis, single-stranded conformation analysis (SSCA), RNase protection assay, allele-specific oligonucleotide (ASO), dot blot analysis, denaturing gradient gel electrophoresis, RT-PCR, quantitative RT-PCR, RFLP and PCR-SSCP. Such assays are known to those of skill in the art.

### 7. METHODS FOR PLANT TRANSFORMATION

Suitable methods for plant transformation for use with the current invention are believed to include virtually any method by which DNA can be introduced into a cell, such as by direct delivery of DNA such as by PEG-mediated transformation of protoplasts, by electroporation, by agitation with silicon carbide fibers, by Agrobacterium-mediated transformation and by acceleration of DNA coated particles, etc. Through the application of techniques such as these, maize cells, as well as those of virtually any other plant species, may be stably transformed, and these cells developed into transgenic plants. In certain embodiments, acceleration methods are preferred and include, for example, microprojectile bombardment and the like. Preferred methods of plant transformation are microprojectile bombardment as illustrated, for example, in U.S. Pat. Nos. 5,015,580; 5,550,318; 5,538,880; 6,160,208; 6,399,861 and 6,403,865 and Agrobacterium-mediated transformation as illustrated, for example, in U.S. Pat. Nos. 5,635,055; 5,824,877; 5,591,616; 5,981,840 and 6,384,301, all of which are incorporated herein by reference.

The seeds of this invention can be harvested from fertile transgenic plants and be used to grow progeny generations of transformed plants of this invention including hybrid plants line comprising the DNA construct operably linked to any of the water-deficit-inducible promoters of the invention.

### 8. RECIPIENT CELLS FOR TRANSFORMATION

Transformation methods of this invention to provide plants comprising an exogenous DNA operably linked to the HVA22, HSP17.5, RAB17, or CA4H promoters are preferably practiced in tissue culture on media and in a controlled environment. “Media” refers to the numerous nutrient mixtures that are used to grow cells in vitro, that is, outside of the intact living organism. A preferred medium is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. The present invention provides techniques for transforming immature embryos and subsequent regeneration of fertile transgenic plants. Practical transformation methods and materials for making transgenic plants of this invention, e.g. various media and recipient target cells, transformation of immature embryos and subsequent regeneration of fertile transgenic plants are disclosed in, for example, U.S. Pat. No. 6,194,636 and U.S. patent application Ser. No. 09/757,089, which are incorporated herein by reference.

### 9. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. As mentioned herein, in order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable are illustrated in, for example, U.S. Pat. Nos. 5,550,318; 5,633,435; 5,780,708 and 6,118,047, all of which are incorporated herein by reference.

Cells that survive the exposure to the selective agent, or cells that have been scored positive in a screening assay, may be cultured in media that supports regeneration of plants. Ideally, seed from the transgenic plant is collected. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

### 10. SITE SPECIFIC INTEGRATION OR EXCISION OF TRANSGENE

It is specifically contemplated by the inventors that one can use techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. This random insertion of introduced DNA into the genome of host cells can be detrimental to the cell if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence.

Site-specific integration can be achieved in plants by means of homologous recombination DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events).

A number of different site specific recombinase systems could be employed in accordance with the instant invention, including, but not limited to, the Cre/lox system of bacteriophage P1, the FLP/FRT system of yeast, the Gin recombinase of phage Mu, the Pin recombinase of E. coli, and the R/RS system of the pSR1 plasmid. The bacteriophage P1 Cre/lox and the yeast FLP/FRT systems constitute two particularly useful systems for site specific integration or excision of transgenes. In these systems, a recombinase (Cre or FLP) will interact specifically with its respective site-specific recombination sequence (lox or FRT, respectively) to invert or excise the intervening sequences. The sequence for each of these two systems is relatively short (34 bp for lox and 47 bp for FRT) and therefore, convenient for use with transformation vectors. The FLP/FRT and Cre/lox recombinase systems have been demonstrated to function efficiently in plant cells. A more thorough discussion of site-specific integration or excision of transgenes may be found in, for example, U.S. Pat. No. 4,959,317 and U.S. Pat. No. 5,527,695, both of which are incorporated herein by reference in their entirety.

### 11. DELETION OF SEQUENCES LOCATED WITHIN THE TRANSGENIC INSERT

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

It is known that homologous recombination results in genetic rearrangements of transgenes in plants. Deletion of sequences by homologous recombination relies upon directly repeated DNA sequences positioned about the region to be excised in which the repeated DNA sequences direct excision utilizing native cellular recombination mechanisms. The first fertile transgenic plants are crossed to produce either hybrid or inbred progeny plants, and from those progeny plants, one or more second fertile transgenic plants are selected which contain a second DNA sequence that has been altered by recombination, preferably resulting in the deletion of the ancillary sequence. The first fertile plant can be either hemizygous or homozygous for the DNA sequence containing the directly repeated DNA which will drive the recombination event.

The directly repeated sequences are located 5′ and 3′ to the target sequence in the transgene. As a result of the recombination event, the transgene target sequence may be deleted, amplified or otherwise modified within the plant genome. In the preferred embodiment, a deletion of the target sequence flanked by the directly repeated sequence will result. See U.S. Pat. No. 6,580,019, incorporated herein by reference in its entirety, for additional discussion of the deletion of sequences located within a transgenic insert.

### 12. BREEDING PLANTS OF THE INVENTION

This invention contemplates both plants directly regenerated from cells which have been transformed with a DNA construct of this invention as well as progeny of such plants, e.g. inbred progeny and hybrid progeny of transformed plants. This invention contemplates transgenic plants produced by direct transformation with a DNA construct of this invention and transgenic plants made by crossing a plant having a construct of the invention to a second plant lacking the construct. Crossing can comprise the following steps:

(a) plant seeds of the first parent plant (e.g. non-transgenic or a transgenic) and a second parent plant having a transgenic DNA construct;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

It is often desirable to introgress a DNA construct into elite varieties, e.g. by backcrossing, to transfer a specific desirable trait from one source to an inbred or other plant that lacks that trait. This can be accomplished, for example, by first crossing a superior inbred (A) (recurrent parent) to a donor inbred (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred, but are like the superior parent for most or almost all other genes. The last backcross generation would be selfed to give progeny which are pure breeding for the gene(s) being transferred, i.e. one or more transformation events.

Therefore, through a series a breeding manipulations, a selected DNA construct may be moved from one line into an entirely different line without the need for further recombinant manipulation. Therefore, one may produce inbred plants which are true breeding for one or more DNA constructs. By crossing different inbred plants, one may produce a large number of different hybrids with different combinations of DNA constructs. In this way, plants may be produced which have the desirable agronomic properties frequently associated with hybrids (“hybrid vigor”), as well as the desirable characteristics imparted by one or more DNA constructs .

Genetic markers may be used to assist in the introgression of one or more DNA constructs of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized. The usefulness of marker assisted selection in breeding transgenic plants of the current invention, as well as types of useful molecular markers, such as but not limited to SSRs and SNPs, are discussed in PCT Application WO 02/062129 and U.S. patent application Nos. 2002133852, 20030049612, and 2003005491 each of which is incorporated herein by reference in their entirety.

The ultimate goal in plant transformation is to produce plants which are useful to man. In this respect, transgenic plants created in accordance with the current invention may be used for virtually any purpose deemed of value to the grower or to the consumer. For example, one may wish to harvest seed for planting purposes, or products may be made from the seed itself such as oil, starch, animal or human food, pharmaceuticals, and various industrial products. Maize is used extensively in the food and feed industries, as well as in industrial applications. Further discussion of the uses of maize can be found, for example, in U.S. Pat. Nos. 6,194,636; 6,207,879; 6,232,526; 6,426,446; 6,429,357; 6,433,252, 6,437,217 and 6,583,338 and PCT Publications WO 95/06128 and WO 02/057471, each of which is specifically incorporated herein by reference in its entirety.

### 13. EXAMPLES

The following examples are included to illustrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

This example illustrates the isolation of Zea mays water-deficit-inducible promoters. Maize lines were field-grown under non-irrigated (water-deficit-producing) or irrigated (well-watered) conditions. Water-deficit conditions were achieved by growing in a geographical location in which rainfall was usually limiting, and, if needed, by withholding irrigation.

Leaf samples were taken from plants before the tassel stage for each condition. Leaf tissue was used to determine water potential. RNA was isolated from the water-deficit (i.e. having a water potential less than about −0.7 MPa) and well-watered samples and was analyzed for differences using transcriptional profiling array methods. A number of RNAs were found to show differences in accumulation, to either higher or lower levels in the plants, depending upon the water treatment. Array samples were selected that demonstrated at least a 3-fold increase in accumulation under water-deficit conditions versus well-watered conditions. Candidate water-deficit-inducible genes were identified as a heat shock protein 17.5 (HSP17.5) gene, a gene encoding an HVA22 protein, a gene encoding cinnamic acid 4-hydroxylase (CA4H) and a gene encoding RAB 17.

Under well-watered conditions, the HSP17.5 gene was found to be expressed to low levels in spikelet, shank, cob and internode tissues; the CA4H gene was expressed to low levels in root and cob tissues. The HVA22 gene, also under well-watered conditions, was found to have no expression in any of the maize tissues tested. As shown in Table 1 under water-deficit conditions, the HSP17.5, RAB17, CA4H and HVA22 genes exhibited about a 3 to 8 fold increase in expression in the sampled leaf tissue.

TABLE 1

| Gene | Measured increased expression |
|---|---|
| HSP17.5 | 4.3 x |
| CA4H | 3.4 x |
| HVA22 | 3.8 x |
| RAB17 | 8 x |

Genome Walker™ technology (CLONTECH, Palo Alto, Calif.) and PCR was used to isolate the 5′ regulatory regions for the maize HVA22, HSP17.5 and CA4H genes. Oligonucleotide primers used in the PCR reactions were designed based upon the gene sequence as identified by the transcriptional array results. PCR was used to isolate the 5′ regulatory regions for the maize RAB17 gene. Gene specific primers for use with the Genome Walker method are shown in Table 2 and primers for PCR of a RAB17 promoter are shown in Table 3.

One skilled in the art would realize that other primers could be designed using the gene coding sequence to obtain similar results. PCR amplified promoters from maize were cloned and sequenced.

TABLE 2

Forward PCR primers for promoter isolation using
Genome Walker

| Gene | Primary PCR Primer | Seq ID |
|---|---|---|
| HVA22 | CCTTGAACTGCGGCAGCGCCAGCCAC | SEQ ID NO:6 |
| (maize) | | |
| HSP17.5 | GAGCTCCTTGACGTCGGCCGGGGTGA | SEQ ID NO:7 |
| (maize) | | |
| CA4H | ACCACCACCAGGTTGCGCACGCCCAT | SEQ ID NO:8 |
| (maize) | | |
| Gene | Secondary PCR Primer | SEQ ID |
| HVA22 | CAGCCACGCCGCGAACAGCACCTTGG | SEQ ID NO:10 |
| (maize) | | |
| HSP17.5 | GTCGCCGTCGGGCACGTCCAGAAGGT | SEQ ID NO:11 |
| (maize) | | |
| CA4H | CGCCATGGCCATCAGGTTGCGGTGGT | SEQ ID NO:12 |
| (maize) | | |

TABLE 3
PCR primers for isolation of a maize RAB17 promoter.

| | | |
|---|---|---|
| Gene | Secondary PCR Primer | SEQ ID |

| | | |
|---|---|---|
| RAB17 (maize) | TATCCAACTCGAGGGTACTCCTGAGATACTATACCC* | SEQ ID NO:9 |
| RAB17 (maize) | GTACTCCATGGTGCTTGCACGGCTTG* | SEQ ID NO: 13 |

*Bold indicates sequences in primer useful for cloning.

### Example 2

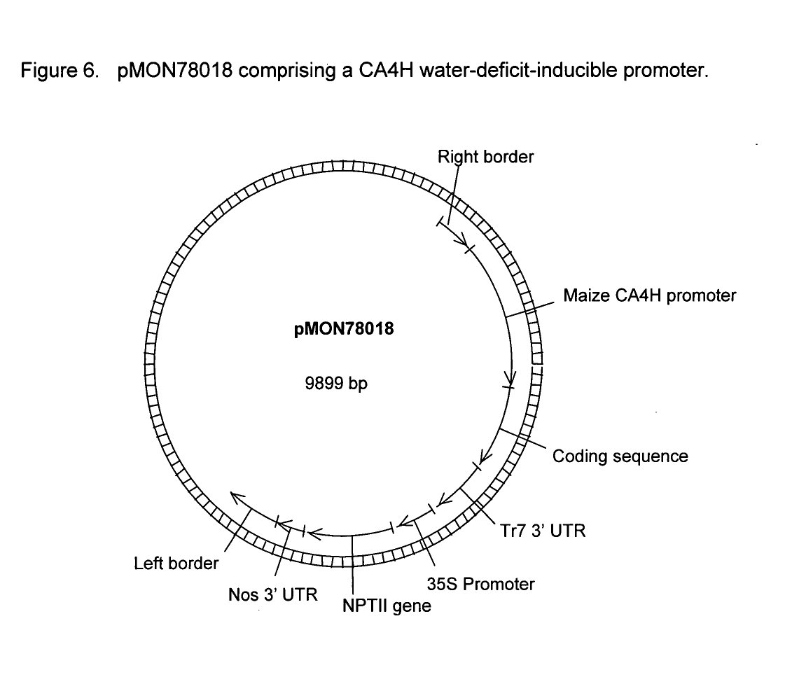

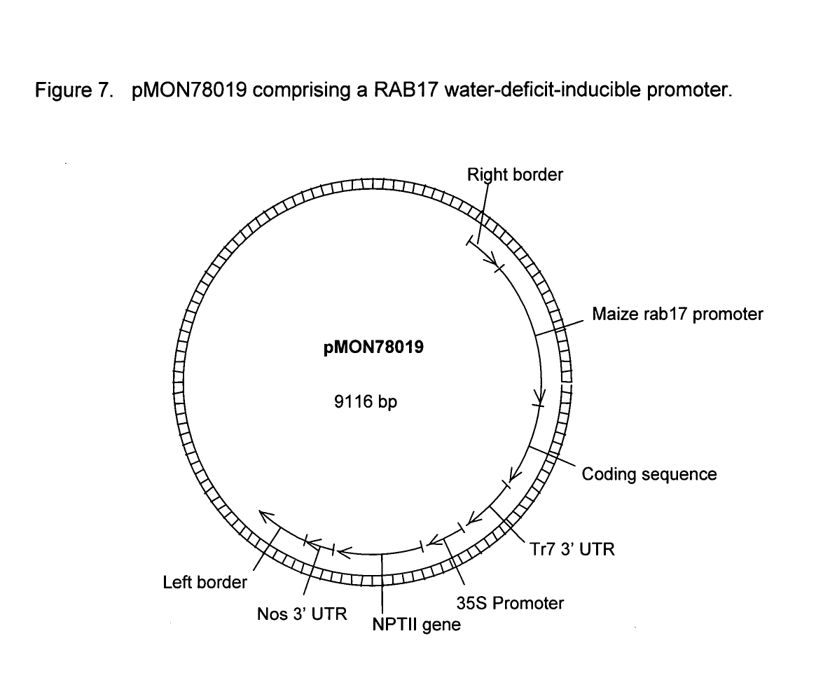

This example illustrates the construction of transformation vectors comprising a water-deficit-inducible 5′ regulatory region derived from the CA4H (SEQ ID NO:3) or RAB17 (SEQ ID NO:5) promoters. In each case, the full length promoter was isolated and operably linked to a heterologous gene for testing promoter activity. See FIGS. 6 and 7 for illustrations of the plasmid vectors used for testing the activity of the maize water-deficit-inducible promoters CA4H (SEQ ID NO:3) or RAB 17 (SEQ ID NO:5).

Any gene operably linked to a water-deficit promoter of the present invention is useful in determining functionality of a promoter. One of ordinary skill knows that there are many ways to monitor promoter activity in a cell, such as by monitoring RNA or protein levels, or by other means of detection such as biochemical or phenotypic observations and the like. Exemplary constructs therefore, comprise, in order from 5′ to 3′, one of the CA4H (SEQ ID NO:3) or RAB17 (SEQ ID NO:5) promoters of the instant invention, or a fragment thereof, operably linked to any gene which is operably linked to a 3′ UTR. Other constructs comprise, in order from 5′ to 3′, one of the CA4H (SEQ ID NO:3) or RAB17 (SEQ ID NO:5) promoters of the instant invention, or a fragment thereof, operably linked to an intron, operably linked to the any gene, operably linked to a 3′ UTR.

Plasmid vectors comprising one of the promoters of the present invention operably linked to a heterologous sequence were used for the transformation of plants. FIGS. 6 and 7 illustrate the plasmid vectors pMON78018 and pMON78019 used for testing the activity of the maize water-deficit-inducible promoters CA4H (SEQ ID NO:3) or RAB17 (SEQ ID NO:5), respectively. Furthermore, isolated fragments of the vectors, especially those comprising the promoter and a selected heterologous DNA are used in transformations experiments.

### Example 3

This example illustrates the transformation of maize with DNA constructs comprising water-deficit-inducible promoters. Agrobacterium tumefaciens-mediated transformation of maize was carried out using vectors pMON78018 or pMON78019. The treated cells were then allowed to recover and regenerated on various media until they were of suitable condition for regeneration into plants, preferably fertile plants.

Several plantlets were produced from transformed maize cells. A number of independent transformation events were tested and assayed for promoter activity by reverse-transcriptase PCR amplification (RT-PCR).

### Example 4

This example describes the water-deficit testing and reverse-transcribed PCR amplification (RT-PCR) assay for determining promoter activity.

Different transformants comprising different insertion events of pMON78018 or pMON78019 were tested for water-deficit response. Plants were sampled at approximately the V4 leaf stage. Leaf disks were punched from each plant and floated upon water or solutions to mimic water-stress or water-deficit: a solution of 100 uM abscisic acid (ABA), or a solution of 250 mM NaCl was used. Following incubation, the disks were retrieved and flash frozen. RNA was extracted from the leaf samples and used to determine the RNA transcribed by the gene and Tr7 3′ UTR operably linked to the RAB17 or CA4H promoters of the invention.

Taqman® assay methods (available from Applied Biosystems, Foster City, Calif.) were used to detect reverse-transcribed PCR amplification products specific for RNA comprising the Tr7 3′ UTR of pMON78018 and pMON78019. Since the Tr7 sequence is not native to maize, any reverse-transcribed amplification products detected represent RNA produced from the exogenous DNA constructs comprising the RAB17 or CA4H promoters and Tr7 3′ UTR. The primers for amplification and probe used for detection were specific for the Tr7 3′ UTR and did not recognize sequence inherent to the maize plant.

As can be seen in Table 4, the maize CA4H (SEQ ID NO:3) and RAB17 (SEQ ID NO:5) promoters of the invention were induced by ABA-solution or salt-solution treatment of the leaf disks. Data are represented as fold increase in accumulation of RNA in the treated samples versus the water control samples, as determined by reverse-transcriptase PCR and detection by Taqmang assay methods (available from Applied Biosystems, Foster City, Calif.). On average, the RAB17 promoter exhibited a 2.71 -fold increase following exposure to 100 uM ABA and a 3.42-fold increase following exposure to 250 mM NaCl. On average, the CA4H promoter exhibited a 2.61-fold increase following exposure to 100 uM ABA and an 8.44-fold increase following exposure to 250 mM NaCl. Thus, the maize RAB17 and CA4H promoters identified by the present inventors are induced in leaf disks of stably transformed plants following treatment of the disks by water-deficit mimicking conditions.

TABLE 4
Fold induction of promoter response

| Construct | Promoter | Event | 100 uM ABA* | 250 mM NaCl* |
|---|---|---|---|---|
| pMON78018 | CA4H | 1 | 3.92 | 19.84 |
| pMON78018 | CA4H | 2 | 1.43 | 10.93 |
| pMON78018 | CA4H | 3 | 3.32 | 6.41 |
| pMON78018 | CA4H | 4 | 1.93 | 2.83 |
| pMON78018 | CA4H | 5 | 2.46 | 2.19 |
| pMON78018 | CA4H | Average | 2.61 | 8.44 |
| pMON78019 | RAB17 | 1 | 4.26 | 4.00 |
| pMON78019 | RAB17 | 2 | 1.48 | 5.35 |
| pMON78019 | RAB17 | 3 | 2.10 | 2.53 |
| pMON78019 | RAB17 | 4 | 2.19 | 3.27 |
| pMON78019 | RAB17 | 5 | 3.53 | 1.96 |
| pMON78019 | RAB17 | Average | 2.71 | 3.42 |

*Fold induction relative to water controls following a four hour treatment.

### Example 5

This example illustrates additional analyses of maize HSP17.5, HVA22, RAB17, or CA4H promoter expression in fertile transgenic maize.

Plants regenerated from a number of independent transformation events are assayed for promoter activity. Activity may be monitored by an RT-PCR assay method or, when a reporter gene such as the uidA gene is used (the protein product of which is commonly referred to as GUS), by histochemical staining. GUS expression patterns, or expression patterns of any exogenous DNA sequence operably linked to a water-deficit-inducible promoter of the invention, are examined in stably transformed R₀ maize plants produced from the transformation procedure, and are also examined in the R₁ generation, or other generations, of transformed plants. A number of different tissues are examined, including but not limited to, leaves, male and female reproductive tissues, and roots. Exogenous DNA expression in transgenic plants, as driven by one of the maize HSP17.5 (SEQ ID NO:1), HVA22 (SEQ ID NO:2), CA4H (SEQ ID NO:3) or RAB17 (SEQ ID NO:5) water-deficit-inducible promoters, is assayed under water-deficit and well-watered conditions. It is expected that expression of exogenous sequences operably linked to a water-deficit-inducible promoter of the invention, will be low or undetectable in transformed plants under well-watered conditions; it is expected that under water-deficit conditions, increased exogenous DNA expression as driven by one of the water-deficit-inducible promoters of the invention will be observed.

Additional expression analysis of the promoters may be carried out. For example, transgenic plants comprising an exogenous DNA operably linked to one of the water-deficit-inducible promoters of the present invention may be subjected to well-watered and water-deficit conditions in a field as described in Example 1. Examination of the expression of the exogenous DNA will provide information as to the expression of the water-deficit-inducible promoter under the test and control conditions.

In a controlled environment such as a greenhouse, water-deficit may be imposed upon the plants using a variety of assay conditions, including but not limited to, germinating seed under water-deficit conditions, or imposing water-deficit conditions on seedlings or plants at any stage of development. Water-deficit is induced by withholding or limiting water, or application of solutions which induce water-deficit conditions such as saline or PEG solutions, or by the application of hormones which induce water-deficit conditions such as ABA.

Any number of parameters may be measured to determine increased tolerance to water-deficit such as measuring plant height, leaf length, number of leaves, root length, root mass, shoot mass, seed set, number of seed, yield, photosynthesis, turgor pressure, osmotic potential, amount of pollen, silking, germination, and the like. In the practice of the current invention, maize plants transformed with a maize water-deficit-inducible promoter operably linked to an exogenous DNA, the product of which is expected to impart increased tolerance to and increased yield under water-deficit conditions.

All of the DNA constructs, transgenic plants and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A transgenic plant having in its genome an exogenous DNA construct comprising a water-deficit-inducible promoter operably linked to a heterologous DNA, wherein said promoter exhibits promoter activity and
a) is derived from about 100 to about 1300 contiguous nucleotides of DNA, wherein said contiguous nucleotides of DNA have from 85% to 100% sequence identity to about 100 to about 1300 contiguous nucleotides of DNA having the sequence of SEQ ID NO:1 or SEQ ID NO:3, or
b) is derived from about 100 to about 760 contiguous nucleotides of DNA, wherein said contiguous nucleotides of DNA have from 85% to 100% sequence identity to about 100 to about 760 contiguous nucleotides of DNA having the sequence of SEQ ID NO:2, or
c) is derived from about 100 to about 570 contiguous nucleotides of DNA, wherein said contiguous nucleotides of DNA have 100% sequence identity to about 100 to about 570 contiguous nucleotides of DNA having the sequence of SEQ ID NO:5 and wherein said contiguous nucleotides comprise nucleotides 156 and 157 of SEQ ID NO:5.

2. A transgenic plant according to claim 1 wherein said promoter comprises DNA with a nucleic acid sequence of SEQ ID NO:1, SEQ ID NO:2, SEQ ID NO:3 or SEQ ID NO:5.

3. A plant according to claim 1 wherein said promoter comprises CAAT and TATA box elements.

4. A plant according to claim 1 wherein said promoter is operably linked to a heterologous DNA which encodes a molecule imparting a plant physiological benefit, pest resistance or disease resistance during water-deficit.

5. A transgenic plant according to claim 1 wherein said heterologous DNA transcribes to RNA imparting gene suppression.

6. A transgenic plant according to claim 5 wherein said heterologous DNA transcribes to double-stranded RNA for suppressing a native gene in a transgenic plant during water-deficit conditions.

7. A plant according to claim 1 selected from the group consisting of corn, soybean, cotton wheat, rice and canola.

8. A plant according to claim 1 wherein said promoter is a water-deficit-inducible promoter which exhibits promoter activity in plant tissue having a water potential of less than −0.7 megaPascals.

9. Seed from a plant of claim 1.

10. A DNA construct comprising a promoter of claim 1 operably linked to a heterologous DNA.

11. A DNA construct according to claim 10 wherein said promoter comprises nucleic acid sequence from SEQ ID NO:1, SEQ ID NO:2, SEQ ID NO:3 or SEQ ID NO:5.

12. A DNA construct according to claim 10 wherein said promoter is a water-deficit-inducible promoter which exhibits promoter activity in plant tissue having a water potential of less than about −0.7 megaPascals.

13. A DNA construct according to claim 10 wherein said promoter exhibits promoter activity in plant tissue having a water potential of less than about −0.8 megaPascals.

14. A DNA construct according to claim 10 wherein said promoter comprises CAAT and TATA box elements.

15. A DNA construct according to claim 10 wherein said promoter is operably linked to a heterologous DNA which encodes a molecule imparting pest or disease resistance or a plant physiological benefit during water-deficit.

16. A method for providing a transgenic plant which produces an RNA of interest in plant tissue in water-deficit comprising introducing into the genome of said plant a DNA construct according to claim 10.

17. A substantially purified DNA having water-deficit-inducible promoter activity in plants wherein said DNA promoter comprises
a) from about 100 to about 1300 contiguous nucleotides of DNA, wherein said contiguous nucleotides of DNA have from 85% to 100% sequence identity to about 100 to about 1300 contiguous nucleotides of DNA having the sequence of SEQ ID NO:1 or SEQ ID NO:3, or
b) from about 100 to about 760 contiguous nucleotides of DNA, wherein said contiguous nucleotides of DNA have from 85% to 100% sequence identity to about 100 to about 760 contiguous nucleotides of DNA having the sequence of SEQ ID NO:2, or
c) from about 100 to about 570 contiguous nucleotides of DNA, wherein said contiguous nucleotides of DNA have 100% sequence identity to about 100 to about 570 contiguous nucleotides of DNA having the sequence of SEQ ID NO:5 and wherein said contiguous nucleotides comprise nucleotides 156 and 157 of SEQ ID NO:5.


SEQUENCE LISTING (13 sequences)

SEQ ID NO: 1 - DNA, 1316 bp, Zea mays
ctgccacatcggcatgtacttagggcgctagctctcccccgctagacacgtagcactctgctacacccctcattgtacacctggatcctcttcttacgcctataaaaggaaggaccaggaccctcttagagagggttggccgcgcggggacgaggacgagacaggcgctctcttggggccgctcgcttccctctcccgcgtggacgcttgtaactccctactgcaagcgcacccgacctgggcgcggggcgaacacaaaggccgcgggattcccacctctctcacgccggtctccggccgcctcgcttctctccccttcgcgctcgccctcgcgctcgacccatctgggctggagcacgcgacgacactcactcgtcggcccaagggaccccccggtctcggaacgcgacactatcttttcacacttagaagctggcaagaaggtcaaacaaataaggtcttatcgtgtatattatttttgcattgcagatagagtggagtttgaaataaaaggtgagatagcaggagtggaaatgggctcaaaaatttatactataaaattgaatgatcaaatcgaattaagatcggactttatttgtattcattcttgaactaaaattatttaactatcataatttattgtggataaacatttggaccacgattcattgccatcgataggaggtgttgtaagagagccagaaagcttaggacatgtaacccgattaaataaagagtcttttgaagtgtccctaagggctacgtgaaaaaaaatcaagagacatactctttgtgaagagtctgtctctacacaaatctctatataagttgtgtctcaattacattattatctagagactcagtgttgtatcacgtagtcttttagtggtctcttttatttgaaatccgttgcagagtcccttatgtgcagagtttggacatcccacgcggtagaagcgacgtggcagttgccacagtatactgacgtgtgggcccagaaaaccccactgtcaatggagaaagaccatccaaagcacagagacttctattttattcgtgactcttccagaatcccgaccatcccacacagagccacggacgcgggacgcctacgcctcgccgcgcccggggccccgcacagtccacagcctttcagaaccttccgtcgccttccagaagaacagaagcccacccgtcgccaccaatataaatcgcccctccagatcggcactccgcacaccaagaatcacatcacacagcgaaccgagaaaccaacacagcaacaagcaaagcagcgatccgacatccgagagatg

SEQ ID NO: 2 - DNA, 764 bp, Zea mays
gtggagtggtggacactagtgccgcggttcatctgacacgtgtcgccacgtgccgccatggcagcacctcagcccggccggcgggccgactgacgtcttgggcaaagcggcgagcgacgcaggcggcgaaagccatccgatttgacccctcgctagacccttcaagaacgaacgctgtgctgctcagatcagaccgtgtctgcctcaaagcgatgccaggacgccacgtccaagcaaagcacccgatgccattgccacctcccagcactcacgcgtgagcgtgactataaaaaacgcaccctctgcatccgcccccgtctgcctgccctaccgaatctttcgccgtcccatcagcccagcaattcttcgctgttcgaggacccctcggtttcgaccgaagcccagcaagccgaccacacaccgctgccgttggttccgtcccaagagatgggcaagtcctggtcgctcatcagccacctccacaccgtcgccgggtagtttcactgttccctcgcagttcgttttccgattcctcctcgtccatctattgggctcgctcgacgctggatgcctgacgtgtgcgtttgctgcttctttgtcttgtctgtccctccctcccgtttcgcaggccaagcatcaccctgctgtaccctctgtaagttccttcatcaccctaataatagcagggaccagttttaccagtgcagcagtgaccgaccacggtccacggcatacgtgagctgagagcatcgtgctgggacatg

SEQ ID NO: 3 - DNA, 1310 bp, Zea mays
aagacttaaaaatcaacaatatcttcacatgacttaattataatgtcttgcttgagacgttgtttttgctactacataagataaagttcaaataaatgcatggtggagttcagcctaggcaaagtgatggtccgaatgattaacaccccaagcaagacattataagtcatgtgaagatctgcaagacgtgctaagagtctctaacacaccaacaagtggaagcccgaacaaacaaaaacgaagccatcaaagttgagataaagaggtggataaattgaaaattgtctcatgattttggatatactcaaatcgacatgacttcatctctaaactatagaacttttgatttgcttttcaaaaagtccaagatcaacaaaacgtgttggtgggtgcgggtttggttcttaacccaataggttttttctcgtgtgtatgaaaaggttgtacccatgtgtgaccgagccagacaggggtacgggcaaaccgaagggaaaccacttaggtggatcccttggctagcctgagactgacacaccataagtgatcggccgcttttaactacgcctggtgccgagccacaatagagatgtcggtctgtctcccacttatgacctacgaacccctcgtactatggctcatctatgggtcgtgtgccccttggcttactgcgcactcatgccctatcaaggctaggccagagtgcgtaggccgctttcagagatcactcggtgaaaaaatcactcggtgatgaaaccggcgaactgtcgttgggtgggtgggtctaactatcaaagaaaacgtattccagcaaacgtattccactctccacaaaataaacatttctgttcggttacctaggtgaggcatcttgtaagaacttggctgtgtttagtcacagcaaacgtattccactctccacaaaataaaataaaaaacgggtcagtgaagctgcaattaatcccttctcttgcttgctggttgctgccagggaaatggcattagtgtttgttcccgttccgaagaccgcagcaacccccggaatcggaaacgcctgccccctgcagcaccaaagaccgtaccaacccccgcaatcgcagttcgcaaaccaaactaatttgtgtacacaaaccggccccgtctcggttctattctataaaacccccgccagaccgctggcttgttccgtcgcctccgctgtccgctgcacagactgtagtaccggggcaggggcaggggcaggggcacaaacagagccacaccacacacagaccccacctacgctacgctacgcgcgtgctgggcgagtgatg

SEQ ID NO: 4 - DNA, 574 bp, Zea mays
gtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctcgtgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatcatg

SEQ ID NO: 5 - DNA, 571 bp, Zea mays
ctcgagggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccatg

SEQ ID NO: 6 - DNA, 26 bp, Artificial
  PCR primer
ccttgaactgcggcagcgccagccac

SEQ ID NO: 7 - DNA, 26 bp, Artificial
  PCR Primer
gagctccttgacgtcggccggggtga

SEQ ID NO: 8 - DNA, 26 bp, Artificial
  PCR Primer
accaccaccaggttgcgcacgcccat

SEQ ID NO: 9 - DNA, 36 bp, Artificial
  PCR Primer
tatccaactcgagggtactcctgagatactataccc

SEQ ID NO: 10 - DNA, 26 bp, Artificial
  PCR Primer
cagccacgccgcgaacagcaccttgg

SEQ ID NO: 11 - DNA, 26 bp, Artificial
  PCR Primer
gtcgccgtcgggcacgtccagaaggt

SEQ ID NO: 12 - DNA, 26 bp, Artificial
  PCR Primer
cgccatggccatcaggttgcggtggt

SEQ ID NO: 13 - DNA, 26 bp, Artificial
  PCR Primer
gtactccatggtgcttgcacggcttg